(19) United States
(12) Patent Application Publication
(10) Pub. No.: US 2017/0340201 A1
(43) Pub. Date: Nov. 30, 2017

(54) DEVICE AND METHOD FOR FIXING A RELATIVE GEOMETRIC POSITION OF AN EYE

(71) Applicant: CARL ZEISS MEDITEC AG, Jena (DE)

(72) Inventor: Michael Stefan RILL, Jena (DE)

(73) Assignee: Carl Zeiss Meditec AG, Jena (DE)

(21) Appl. No.: 15/534,260

(22) PCT Filed: Dec. 8, 2015
(86) PCT No.: PCT/EP2015/078992

§ 371 Date: Jun. 8, 2017

(30) Foreign Application Priority Data
Dec. 11, 2014 (DE) 10 2014 225 636.4

(51) Int. Cl. A61B 3/107; A61B 3/10; A61F 9/009; A61B 3/00

(52) U.S. Cl. CPC A61B 3/107; A61F 9/009; A61B 3/102; A61B 3/0083

(57) ABSTRACT

A device and a method for fixing a relative geometric position of an eye with respect to an ophthalmological diagnosis and/or therapy system. A device having a container with upper edge designed to produce a mechanically fixed connection between the container and the ophthalmological diagnosis and/or therapy system, and a lower edge which can be placed with a form fit onto the eye, and a clamping mechanism for producing a force-fit connection of the container to the eye, wherein the clamping mechanism has a movable connection to the container is a fixed rotation point or a fixed rotation axis in a jacket area of the container, and by a corresponding method.

### RELATED APPLICATIONS

This application is a National Phase entry of PCT Application No. PCT/EP2015/078992 filed Dec. 8, 2015 which application claims the benefit of priority to German Application No. 10 2014 225 636.4, filed Dec. 11, 2014, the entire disclosures of which are incorporated herein by reference.

### FIELD OF THE INVENTION

Embodiments of the present invention relate to a device for fixing a relative geometric position of an eye for an ophthalmological diagnosis and/or therapy system. The invention further relates to an ophthalmological diagnosis and/or therapy system with such a device as well as a method for fixing a relative geometric position of an eye for an ophthalmological diagnosis and/or therapy system.

### BACKGROUND

Many examinations, therapies and in particular surgical interventions in ophthalmology are carried out with the assistance of partially or fully automated systems. Thus eye ailments are for example characterized by means of the corresponding diagnostic methods and systems, such as optical coherence tomography (OCT) or ophthalmometric measurements, and subsequently treated by laser-supported surgical operation systems. Ideally, during the therapy physicians draw on previously gathered data and/or additional data are determined. In the process, in many therapeutic interventions a secure positioning of the eye to the intervening medium, such as, for example a laser is of crucial importance.

Among surgical interventions in ophthalmology, refractive surgery, e.g. for the correction of corneal curvature, or cataract surgery are among the most frequent therapeutic methods being carried out. Developments in these areas, which support the secure performance of highly automated, laser-supported operations, benefit other areas of ophthalmology, for which said developments can also be used.

In the case of laser-supported therapeutic methods the relative geometric position of the patient's eye to the laser focus must be precisely defined. The more securely and precisely one can fix said relative geometric position of the patient's eye, the gentler the procedure, because in the case of defined radiation, damages in critical regions of the eye can be avoided.

Since in laser therapy methods local anesthesia is administered to the patient's eye with eye drops, during therapy the patient can move his eye consciously or unconsciously. So-called microsaccades are examples of unconscious movements. These microsaccades are rapid, jerky movements of the eye with very small amplitude that cannot be suppressed through conscious action. Therefore, to fix the relative positioning of the eye and laser system, the patient's eye is “docked” to the laser system with the assistance of a patient interface that is, a device for fixing a relative geometric position of an eye to an ophthalmological diagnosis and/or therapy system, used to establish a connection between the eye and an ophthalmological diagnosis and/or therapy system.

In cataract surgery so-called fluid interfaces are frequently employed. They consist of a funnel-shaped container which is, as a rule, filled with a salt solution (balanced salt solution, BSS). The distal end of the laser optics is subsequently dipped into the salt solution. This arrangement has a number of advantages over contact glasses, which are placed directly on the eye, for example there is only a slight increase of the intraocular pressure and the prevention of the formation of corneal folds, which as a rule lead to disturbance of the optical imaging of the laser focus in the eye lens. In addition, with a fluid interface the adjustment of the refractive index between the laser optic and the corneal material is easily possible.

In contrast, other ophthalmological methods, in particular also other surgical methods such as refractive surgery, use contact glasses.

To be able to fix a relative geometrical position of the eye to be treated to an ophthalmological diagnosis and/or therapy system, patient interfaces must be fixed on the eye. Ordinarily this fixation happens by application of a suctioning of the patient interface on the eye by a vacuum. However, suctioning a patient interface on the eye with a vacuum involves both technical and medical disadvantages: vigorous suctioning, which is necessary for solutions known thus far, in order to actually keep the eye still, sometimes causes heavy bleeding in the conjunctiva. While these injuries heal within a couple of days, both patients and physicians find them to be unpleasant and disruptive. In addition, with vacuum suctioning there is always the risk of a loss of suction, so-called “suction loss”, in which case the patient interface loosens from the eye during the laser procedure. Under circumstances, this can cause serious injuries to the eye, in particular when the laser beam is not disconnected or blocked in time.

Not least, the suctioning of a patient interface on the eye by a vacuum complicates the procedure as well as the system: To generate the vacuum, in particular in the case of laser-supported surgical procedures, in which a manual generation of a vacuum would be too risky, a pump must be integrated into the system and a hose line installed to the patient interface. This increases production costs of the therapy system and a hose connection can also hamper the handling of the therapy system during the operation.

Therefore, it is very advantageous to be able to mechanically fix a patient interface on the eye to be treated without vacuum suctioning. One possibility for a very simple mechanical fixation is revealed in CN 102935025. A contact glass is provided with two hooks which are then strapped with connection belts to an eye speculum. However, such a device does not permit a secure fixation of the eye, since they do not enable the setting of a defined and reproducible contact pressure of the contact glass on the eye.

WO 03/053229 A2 in turn describes an eye speculum that stabilizes the eye through a design as a cast, flexible shell that surrounds the eyeball. In one embodiment it can also be filled with a fluid. However, if such an eye speculum is supposed to be used for fixation of the eye in the sense of a fixing of its geometric position with respect to a diagnosis and/or therapy system, this can only occur through a corresponding pressure on the eye ball, which in turn would lead to an undesired high intraocular pressure. In addition, the eyeball in this case, together with the eye speculum, continues to be movable within limits, so that these movements would have an effect on the ophthalmological diagnosis and/or therapy system.

In order to prevent or at least minimize movements of the eye, in U.S. 2014/0275751 A1 an adhesive effect is produced between a patient interface and the eye to be treated by manufacturing the patient interface out of a collagen-based material. To establish a connection to a therapy system, in addition a fastening ring is affixed to the originally flat patient interface. Optionally, the collagen-based interface is clamped under a conventional eye speculum.

However, due to the design of the patient interface and because the eye speculum can be seated anywhere, a reproducible pressing of the patient interface is difficult. In addition, two hands are required for a corresponding positioning on the eye.

In US 2012/0316544 A1 on the other hand, an interface for ophthalmic surgical systems, in particular a system used in cataract surgery, is described, comprising a container which can contain a solid material or can be filled with a fluid material. The container has a circumferential, at least partially inclined lateral wall. The container is clamped to a conventional eye speculum on this inclined lateral wall and hence pressed against the eye. The inclination of the lateral wall in the process determines the contact pressure. Thus, a fixation of the eye is possible. However, the fact that the eye speculum can be placed anywhere on the lateral wall is critical for the reproducibility of the pressing. In addition, two hands are required for a corresponding positioning.

### SUMMARY OF THE INVENTION

Therefore, example embodiments of the invention address the problem of providing a device for fixation of a relative geometric position of an eye to an ophthalmological diagnosis and/or therapy system that permits a secure mechanical fixation of this device on the eye to be treated without vacuum suction and with a reproducible and adjustable contact pressure, and which makes simple handling possible during placement on the eye. Further, the invention addresses the problem of describing an ophthalmological diagnosis and/or therapy system with such an inventive device as well as a corresponding method for fixing a relative geometric position of an eye to an ophthalmological diagnosis and/or therapy system.

A device for fixing a relative geometric position of an eye to an ophthalmological diagnosis and/or therapy system contains a container with an upper edge that is designed to produce a mechanical fixed connection between the container and the ophthalmological diagnosis and/or therapy system, and a lower edge which can be placed with a form fit on the eye. For the purpose of the invention, a container is a body that can assume any shape in the space, but has at least one opening on each of two sides opposed to each other. The above described upper edge and the above described lower edge run along these openings. The entire outer layer of the container between these two openings is designated as a jacket area.

The container can differ widely in its configuration with regard to its size as well as its material. Depending on its use, it can for example contain a base with which a predominant part of an eye can be covered, however extending only a few millimeters in height, or with the same base having a height of several centimeters. The container can be manufactured of a material and a structure that are permeable to fluid. However, it can also have additional openings or an open structure in the jacket area. Especially well suited as container material or for configuring at least a lower region of the container comprising the lower edge are materials that are at least slightly elastic or reversibly deformable materials such as, for example silicone.

In one preferred embodiment the container is cylindrical or funnel-shaped with a round or oval base.

The device further contains a clamping mechanism for producing a frictionally engaged coupling of the container to the eye. This clamping mechanism is in turn variable in its shape, size and material used for it provided it contains parts that make possible a clamping function through a corresponding reversibly deformability, and/or the possibility is given of mobility of at least two parts of the clamping mechanism relative to one another.

According to the invention, this clamping mechanism has a movable connection to the container via a fixed rotation point or a fixed rotation axis arranged in the jacket area of the container. Such a rotation point can be realized for example by a pin arranged in the jacket area of the container which serves as a rotation axis, or by a hinge arranged in the jacket area of the container which serves as a rotation point. Of course, several fixed rotation points or rotation axes can also be arranged in the jacket area. In one preferred embodiment two such rotation points or rotation axes are arranged opposite one another in the jacket area.

With such an inventive device it is now possible to establish a connection between an eye and an ophthalmological diagnosis and/or therapy system. As a rule, this connection takes place mechanically, for example by a screw connection, by clamping or by suctioning on the upper edge of the device. If the device is fixed on the eye by use of the frictionally engaged coupling by the clamping mechanism, the relative geometric position of an eye to the ophthalmological diagnosis and/or therapy system is thus fixed.

The inventive device has a number of advantages: Avoiding suctioning by application of a vacuum means the conjunctiva is not injured or only slightly injured; and the risk of a suction loss that can lead to eye injuries if a therapy system being operated at that time is not immediately turned off, is averted. The structure of the system is significantly simplified by the fact that hose connections to a vacuum device such as, for example a vacuum pump are avoided. It is also not necessary to have a vacuum pump available. The inventive device is easy to handle; the existing movable connection of the container to the clamping mechanism makes it possible to operate with one hand. In addition, the use of an additional eye speculum is also omitted, since the clamping mechanism can be used in its place.

In one example embodiment the device is further characterized by a detachability of the movable connection of the clamping mechanism to the container. A further example embodiment in the process enables a non-destructive detachability of this connection. In this case, “non-destructive” means that the clamping mechanism and/or the container are preserved in their integrity. Thus, they can be reused and in addition do not constitute a source of particles. This is possible, for example by a screw connection or clamp connection. The latter is made possible by a correspondingly flexible material by placement onto a pin acting as a rotation point or a hinge ball acting as a rotation point. However, a design is also conceivable in which a rotation point or a rotation axis is realized by a pin or hinge ball located on the clamping mechanism which can be inserted into a hollow cylinder or a hinge socket if necessary with slight pressure in an opening of the hollow cylinder or hinge socket provided for this purpose. This offers the advantage that after the appropriate steps of a therapy in which the diagnosis and/or therapy system is used, it is possible to continue working on the eye that has had the container removed, but the clamping mechanism remains on the eye and for example continues to be used there as a simple eye speculum.

It is advantageous if the clamping mechanism contains two mutually tensible straps for fixing the eye. On the one hand, this enables a stable design. On the other hand, it makes possible a very good dosability of the force that is applied in order to establish the frictionally engaged coupling of the container to the eye.

In principle, these straps are variable in their shape and size as well as in the material to be used, however with the restriction that it must be possible to arrange a part of the strap between the eyeball and the eyelid. Also, the material must be compatible for direct contact with the eye tissue.

A device containing a clamping mechanism with two mutually tensible straps that are connected to one another via a spring coupling embodies in turn a special design of the inventive device that makes especially easy a tensioning of the clamping mechanism with simultaneous control of the positioning of the container on the eye.

The frictionally engaged coupling of the container to the eye is produced in the inventive device by the clamping effect of the clamping mechanism, which is partly placed between the eyeball and the eyelid. In one especially advantageous embodiment of the inventive device the clamping mechanism has structure for blocking the eyelid. This is the case when the part of the clamping mechanism that is placed between the eyeball and the eyelid is designed such that it can grip over a predominant part of the width of an eye between the eyeball and the lid and thus hold back the entire lid in its width and prevent it from closing. The structure for blocking the lids can in the process be realized either by having the straps of the clamping mechanism themselves correspondingly molded or can contain a special attachment.

In principle, a closing of the eyelids is also prevented when the clamping mechanism grips only at a point between the eyeball and eyelid. However, this is more unpleasant for the patient and leads to an unfavorable clamping point or pressure point on the eye. Furthermore, a clamping mechanism that only grips at a point between the eyeball and the lid also does not ensure a secure opening of the eye, for example to permit the eye of the patient to be worked on manually after removal of the container.

A further example embodiment of the inventive device permits the reduction of the contact pressure exerted on the eyeball by the clamping mechanism: This is the case when the lower edge of the container has an eye contact surface that is designed to produce an adhesion effect between this eye contact surface and the eye and thus without a clamping action of the device to actively minimize the relative movement of the eye to the container. With such a designed eye contact surface a relative movement of the eye to the container is ultimately prevented when, through the clamping mechanism the frictionally engaged coupling of the container to the eye is only produced with a reduced contact pressure. Such an adhesion effect within the scope of this application in the process comprises an actual increase of an adhesion force chemically as well as also an increase of the friction force between the eye contact surface and the eye mechanically.

In particular, such an embodiment of the inventive device can have an eye contact surface on the lower edge of the container which contains nubs that can be indented into the conjunctiva of the eye. In this connection, a plurality of shapes of the nubs are conceivable, such as, for example a truncated cone, pointed cone, a truncated pyramid, a pointed pyramid, thin pins or needles.

As an alternative or at the same time, it is possible to subject the corresponding eye contact surface of the device to a surface treatment with which the surface roughness is increased, or to provide the surface of this eye contact surface with an adhesion-active surface coating. With such an adhesion-active surface coating by chemical interaction through the construction of corresponding bonds, or for example, by dehydration of the cornea initiated by the coating and thus of a temporary drying of the cornea or the conjunctiva below the eye contact surface of the device it is possible to achieve an increased adhesion force or also friction force.

The container of the inventive device is furthermore may also be designed to accommodate a refractive acting material in at least a part of the volume range between the cornea of the eye and the ophthalmological diagnosis and/or therapy system. By refractive acting material, a material is meant here of any aggregate state that has a refractive index unequal to the refractive index of a medium surrounding this material. Depending on the scope of application and type of refractive acting material, this means for example that the jacket is correspondingly configured and/or corresponding contact edges or surfaces are provided near the lower edge, thus the container does not require any additional processing or other preparations, in order to accommodate a refractive acting material for the case of application.

Thus the container can be designed to accommodate a contact glass in one example inventive configuration. For example, this enables a use of the inventive device in laser-supported surgery, but also in non-invasive therapy methods.

However, in a further inventive embodiment the container can also be designed to be filled with a refractive acting fluid or a refractive acting gel. This makes the inventive device interesting, for example for use in cataract surgery.

In the process, in one special embodiment a combination of the accommodation of an optical lens or a contact-glass similar device as well as a subsequent filling with a refractive acting fluid or a refractive acting gel are possible.

Depending on the design of the lower edge of the container and any filling of the container, in one further example embodiment of the present invention a sealing ring extends along the lower edge of the container. With this, a tight connection between the eye and the container can be produced for any refractive acting material, and for example, in one case relief is provided for a possible discharge of a refractive acting fluid by the fact that through one design of an eye contact surface on the lower edge of the container through nubs there is no corresponding impermeability between the container and the eye for the refractive acting fluid even after establishment of the frictionally engaged coupling of the container to the eye.

The inventive device for fixing a relative geometric position of an eye to an ophthalmological diagnosis and/or therapy system is furthermore part of an inventive diagnosis and/or therapy system. Through the establishment of a mechanically fixed connection between the container and the ophthalmological diagnosis and/or therapy system, as soon as a frictionally engaged coupling between the container of the device and the eye has occurred which can occur either prior to the establishment of a mechanically fixed connection between the container and the ophthalmological diagnosis and/or therapy system or afterwards the relative geometric position of the eye to the ophthalmological diagnosis and/or therapy system can no longer be changed and is thus fixed.

The following steps are included in an inventive method for fixing a relative geometric position of an eye to an ophthalmological diagnosis and/or therapy system:

An inventive device for fixing a relative geometric position of an eye to an ophthalmological diagnosis and/or therapy system is placed on an eye after the clamping mechanism of the device has been brought to a first position. This first position is characterized by the fact that the parts of the clamping mechanism facing the eye are approximated in their location to one another and hence can be placed unimpeded on an eye. Such an approximation can, for example take place as a result of an exterior compressive force having to be exerted on parts of the clamping mechanism.

After placement on the eye, the device for fixing a relative geometric position of an eye is manually or automatically aligned on an optical axis of the eye. To this end, the clamping mechanism must continue to be held in the first position. Thus, if this first position requires the exertion of an exterior compressive force, it must be maintained during the alignment.

As a rule, a manual alignment occurs by shifting the device on the eye by a person who is carrying out the corresponding diagnosis or therapy. The manual alignment can be assisted by corresponding analysis methods such as, for example a microscope or a slit lamp which is directed on the device located on the eye. However, an automatic alignment is also conceivable, in which the device is automatically brought into line with the desired position on the eye, wherein this can occur with the assistance of image detection methods or other assistance systems.

Once the desired position of the device on the eye has been achieved, the clamping mechanism of the device is brought to a second position. In this second position the distance of the parts of the clamping mechanism facing the eye to one another is increased over the first position such that these parts of the clamping mechanism are arranged between an eyeball of the eye and a lid of the eye, wherein a frictionally engaged coupling to the eye results. Such a transition from a first to a second position occurs for example by incremental withdrawal of an exterior compressive force, which causes a sliding of the parts from the central eye region to the region between the eyeball and the eyelid. In the process, the relevant parts of the clamping mechanism gain purchase on the eye-side lid tissue, which generates a corresponding contact pressure of the container which is moveably connected to the clamping mechanism on the eye. With this, the container is fixed on the eye in such a way that said eye can no longer perform movements on its own.

Once the relative geometric position of the ophthalmological diagnosis and/or therapy system to the inventive device has been determined, the relative geometric position of an eye to the ophthalmological diagnosis and/or therapy system has also been fixed.

The relative position of the ophthalmological diagnosis and/or therapy system of the inventive device can be produced by a corresponding mechanical fixed connection of the device via the upper edge of the container of this device to the ophthalmological diagnosis and/or therapy system prior to, between, or after the above mentioned steps of the method.

Such a connection can occur mechanically for example by screwing, clamping or by suctioning on the upper edge of the device.

### BRIEF DESCRIPTION OF THE DRAWINGS

The present device will now be described with the assistance of exemplary embodiments. The figures show the following:

FIG. 1 depicts a top view of a first embodiment of the inventive device for fixing a relative geometric position of an eye to an ophthalmological diagnosis and/or therapy system;

FIG. 2A depicts a lateral view of a first embodiment and FIG. 2b shows an enlarged section of an eye contact surface of the lower edge of the container of the device;

FIG. 3A depicts a second embodiment of the inventive device in a lateral view and FIG. 3B depicts an enlarged section of a contact surface of the lower edge of the container of the device;

FIG. 4 depicts a schematized comprehensive view of an embodiment of the inventive ophthalmological diagnosis and/or therapy system.

### DETAILED DESCRIPTION

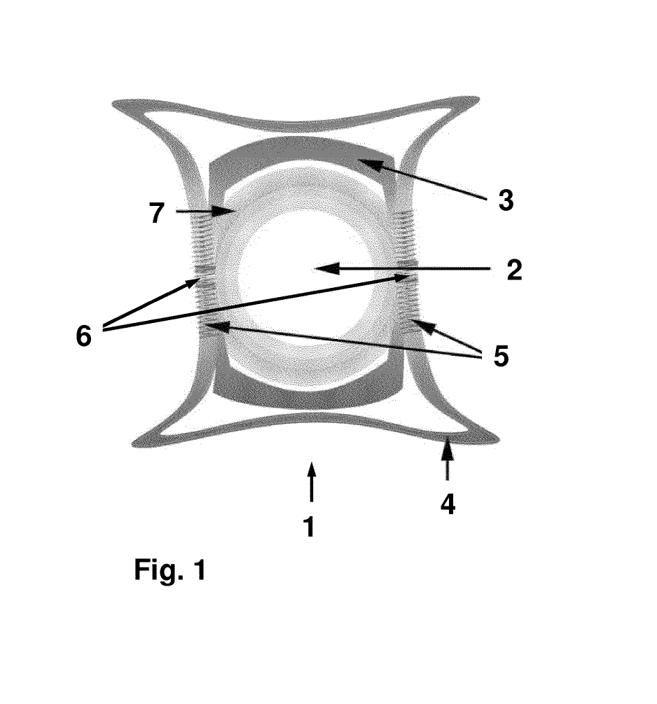

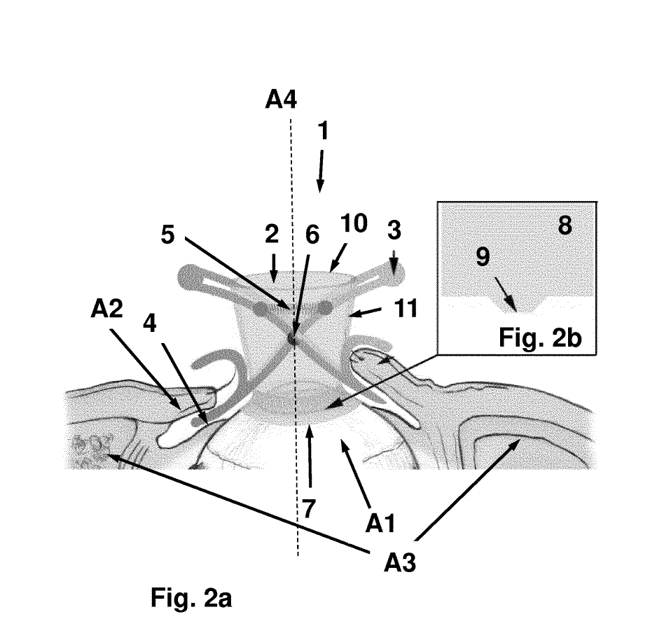

FIG. 1 depicts the top view of a first embodiment of the inventive device for fixing a relative geometric position of an eye to an ophthalmological diagnosis and/or therapy system 1, while FIG. 2A, on the other hand, shows a lateral view of this first embodiment. FIG. 2B shows an enlarged section of an eye contact surface 9 of the lower edge 8 of the container 2 of the device 1.

In this first example embodiment of the inventive device for fixing a relative geometric position 1 of an eye A1 to an ophthalmological diagnosis and/or therapy system a container 2, which can be filled with a fluid and thus can also be referred to as a fluid patient interface, is combined with a clamping mechanism 3, which has the function of an eye speculum.

The container 2 is configured in the shape of a funnel for accommodation of a refractive acting fluid and also contains an opening on its side to the eye A1, as a result of which a lower edge 8 is formed, as well as an opening on the side opposing the diagnosis and/or therapy system, as a result of which an upper edge 10 is formed.

The lower edge 8 is formed and designed such that it can be placed with a form fit on the eye A1. In order to prevent a mechanical shifting of the container 2 on the eye A1 in the pressed state, but simultaneously keep the contact pressure of the container 2 on the eye A1 as low as possible, the lower edge of the container 2 pointing at the eye A1 has an eye contact surface 9, which has nub structures in the form of a plurality of truncated cones and whose surface is roughened.

By pressing the container 2 on the eye A1, the nub structures of the eye contact surface 9 are pressed into the soft conjunctival tissue above the sclera of the eye A1. The friction of the rough surface on the eye surface additionally prevents the entire container 2 from being able to be moved laterally in the eye A1 or prevents the eye A1 of a patient to be treated from being able to be moved consciously or unconsciously.

As a rule, pressing the container 2 on the eye A1 already establishes a sealed, that is, liquid impermeable connection of the container 2 to the eye A1. On the lower edge of the container 2 containing a refractive acting fluid, that is, towards the eye A1, there is, in addition a sealing ring 7. It extends along the entire lower edge 8 of the container 2 and reliably prevents the discharge of the refractive acting fluid: Due to the natural variations of the size of the eye A1 from patient to patient, there could be a corresponding leak in the case of an unfavorable size of the eye A1 that deviates greatly from the norm when, due to the deviating sizes the form fit of the lower edge 8 of the container 2 to the eye A1 does not result in a liquid impermeable connection of the container 2 to the eye A1 by itself.

The upper edge 10 of the container 2 is, in turn designed such that it can be fixed on a diagnosis and/or therapy system by suctioning of this upper edge region. The container 2 is formed of a polymer or another solid material. The jacket region 11 is thus sealed from the refractive acting fluid.

The clamping mechanism 3 is moveably connected to the container 2 via two opposing fixed rotation axes 6 in the jacket region 11 of the container 2. This connection is realized via two pins located on the outside of this funnel-shaped container 2. Two straps 4 of the clamping mechanism 3 acting as an eye speculum are fastened to these pins. The two straps 4 are mechanically connected to one another via a spring coupling 5 by use of a spring exerting compression stress.

In the “docking”, that is, the placement of the container 2, which corresponds to a fluid patient interface, on an eye A1 the straps 4 are pressed together. In the process, the container 2 is aligned with respect to structures of the eye A1, such as, for example the corneal limbus and blood vessels. With this, the container 2 is simultaneously also aligned to the optical axis A4 of the eye. An image detection method can be used for this purpose. When the desired position of the container 2 has been found, the straps 4 are released. In principle, the desired position can also occur subsequently through manual shifting in the clamping state. However, this requires overcoming the retaining force. In the process, the spring presses the straps 4 apart such that their eye-side ends are brought between eyelid A2 and the conjunctiva of the eye A1, as can be seen in the lateral view of FIG. 2a. As a result, the eyelids A2 are opened and held in the opened position. In addition, the pressing apart of the straps 4 also causes the container to be pressed onto the eye A1.

If, after the corresponding treatment steps of the ophthalmological diagnosis and/or therapy system, in which the relative geometric position of an eye A1 to an ophthalmological diagnosis and/or therapy system must be fixed, additional steps follow, in which case the eye A1 must be held open, but a fixing of the eye movement is no longer necessary, the container 2 is pressed together by application of an outer pressure on the jacket area 11 of the container 2, which is configured weakly elastic to this end, such that the pins serving as a rotation axis 6 on the jacket area 11 of the container 2 leave the corresponding counterpart on the clamping mechanism 3 and the container 2 can be pulled upward, i.e. away from the eye, out of the clamping mechanism 3. The clamping mechanism 3, on the other hand remains in place as an eye speculum, so that the eye A1 continues to be held open.

Since eye speculums are quite frequently used in addition to patient interfaces, which are pressed on the eye A1 by application of a vacuum, this embodiment of the inventive device 1 offers not only the advantage of a simpler production of the contact pressure and thus a simplification of the device and a simplified handling of such a device, but rather it also makes the use of an additional eye speculum during the entire treatment of the patient superfluous.

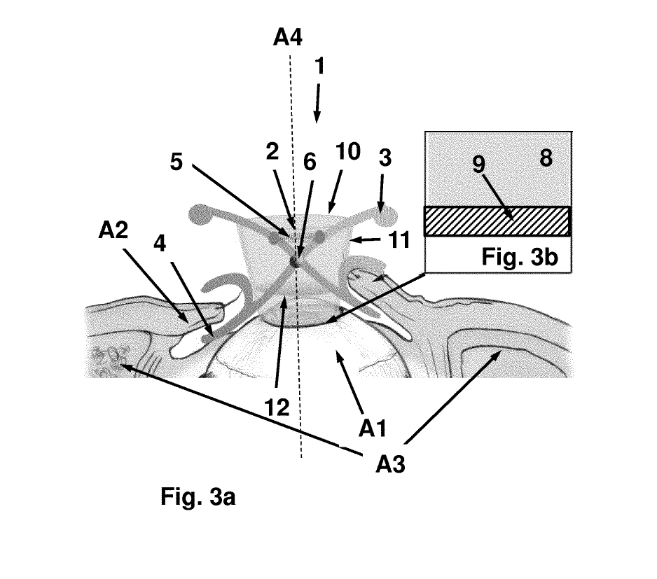

FIG. 3A shows a second embodiment of the inventive device 1 in a lateral view. FIG. 3B, contained within, shows an enlarged section of an eye contact surface 9 of the lower edge 8 of the container 2 of the device 1. In the description of this second embodiment of the inventive device reference is made to the description of the first embodiment and in the following we only go into the differences of the second embodiment to this first one.

In contrast to the first embodiment, the container 2 in the second embodiment is designed to accommodate a contact glass 12. Thus, the container 2 does not have to be liquid impermeable, nor does it have to lie in a liquid impermeable manner on the eye A1. Therefore, in the second embodiment of the present invention it is designed significantly flatter for accommodation of the contact glass 12 as is the case in the first embodiment for accommodation of the refractive acting fluid. In addition, in the second embodiment an additional sealing ring 7 can be dispensed with, since there is no danger of a leak.

The eye contact surface 9 in the second embodiment does not contain any nubs or similar structures. In contrast, it is surface coated with a material that produces an additional adhesion effect between the container 2 and the eye A1. One example of this is a coating with a collagen or a hydrophilic material with adhesive effect that can absorb the tear fluids.

In the process, it is to be noted that the design of the eye contact surface 9 is independent from the refractive active material that is used, so that an inventive device 1 as described in the first embodiment can also contain an adhesion effective surface coating in place of the nub structure and vice versa. Also, a combination of a corresponding nub structure and a surface coating is possible.

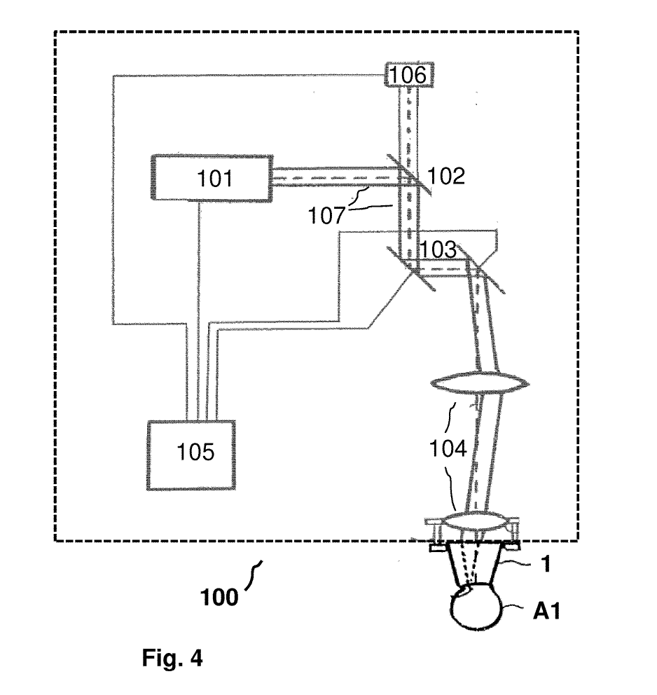

FIG. 4 shows a schematized comprehensive view of an embodiment of the inventive ophthalmological diagnosis and/or therapy system 100. We will not go into the details of such a diagnosis and/or therapy system 100. FIG. 4 only serves the purpose of using the example of a laser therapy system operating according to the LASIK method to show how an eye A1 is fixed on an ophthalmological diagnosis and/or therapy system by use of an inventive device 1 and thus the relative geometric position of an eye A1 is fixed to an ophthalmological diagnosis and/or therapy system 100. The laser therapy system contains a laser 101, whose beam is conducted by a beam splitter 102 to a scanning unit 103, which contains two pivoted scanning mirrors perpendicular to one another. As a result the laser beam can be refracted two-dimensionally. The beam is further conducted to the eye A1 via a projection lens system 104 containing two lenses. A part of the beam is backscattered from the cornea of the eye A1, passes through the beam splitter 102 and is detected by the detector 106. A control unit 105 evaluates the data supplied by the detector 106 and controls the laser 102, the scanning unit 103 and the projection lens system 104. Since the position of the eye A1 to the laser therapy system 100 is fixed by the device for fixing a relative geometric position 1 of an eye A1, it is facilitated that, for example, in a laser surgery application laser irradiation which leaves the ophthalmological diagnosis and/or therapy system can act on the place provided for it on the eye A1 and, on the other hand, signals which are received by the eye A1 in the ophthalmological diagnosis and therapy system can be unambiguously assigned to a place on the eye A1. This is illustrated by the schematic beam projection 107 within the ophthalmological diagnosis and/or therapy system 100.

The foregoing mentioned features that have been explained in various example embodiments can be used not only in the combinations cited by way of example, but rather, can also be used in other combinations or alone, without abandoning the scope of the present invention.

A description related to features of the device applies analogously with respect to these features, while features of the method present corresponding functional features of the described device.

1-14. (canceled)

15. A device for fixing a relative geometric position of an eye with relation to an ophthalmological diagnosis and/or therapy system, the device comprising:
a container having an upper edge that is structured to produce a mechanical fixed connection between the container and the ophthalmological diagnosis and/or therapy system, and a lower edge, which can be placed with a form fit on the eye,
a clamping mechanism that establishes a frictionally engaged coupling of the container to the eye.
wherein
the clamping mechanism includes a movable connection to the container via a fixed rotation point or a fixed rotation axis arranged in jacket area of the container.

16. The device according to claim 1, wherein the, movable connection of the clamping mechanism to the container is detachable.

17. The device according to claim 1, wherein the clamping mechanism further comprises two mutually tensible straps.

18. The device according to claim 3, further comprising a spring coupling the two straps.

19. The device according to claim 1, wherein the clamping mechanism further comprises structure for blocking the lids of the eye from closing.

20. The device according to claim 1, wherein a lower edge of the container further comprises an eye contact surface that is structured to produce an adhesion effect that minimizes a relative movement between the eye and the container when the frictionally engaged coupling of the container to the eye is produced by the clamping mechanism.

21. The device according to claim 6, wherein the eye contact surface comprises nubs that can be indented into a conjunctiva of the eye.

22. The device according to claim 6, further comprising an adhesion-active surface coating of the eye contact surface or a surface treatment of the eye contact surface to increase surface roughness of the eye contact surface.

23. The device according to claim 7, further comprising an adhesion-active surface coating of the eye contact surface or a surface treatment of the eye contact surface to increase surface roughness of the eye contact surface.

24. The device according to claim 1, wherein the container is structured to accommodate a refractive acting material in at least a part of a volume range between a cornea, of the eye and the ophthalmological diagnosis and/or therapy system.

25. The device according to claim 9, wherein the device is further structured to accommodate a contact glass.

26. The device according to claim 9, wherein the device is structured for filling with a refractive acting fluid or a refractive acting gel.

10. The device according to claim 10, wherein the device is structured for filling with a refractive acting fluid or a refractive acting gel.

28. The device according to claim 1, further comprising a sealing ring that extends along the lower edge of the container.

29. An ophthalmological diagnosis and/or therapy system, comprising a device for fixing a relative geometric position of an eye to the ophthalmological diagnosis and/or therapy system, the device comprising:
a container having an upper edge that is structured to produce a mechanical fixed connection between the container and the ophthalmological diagnosis and/or therapy system, and a lower edge, which can be placed with a form fit on the eye,
a clamping mechanism that establishes a frictionally engaged coupling of the container to the eye,
wherein
the damping mechanism includes a movable connection to the container via a fixed rotation point or a fixed rotation axis arranged in a jacket area of the container.

30. A method for fixing a relative geometric position of an eye to an ophthalmological diagnosis and/or therapy system, the method comprising:
placing a device comprising a container with an upper edge that is structured to produce a mechanical fixed connection between the container and the ophthalmological diagnosis and/or therapy system on the ophthalmological diagnosis and/or therapy system;
placing a lower edge of the container with a form fit on the eye;
applying a clamping mechanism to establish a frictionally engaged coupling of the container to the eye by operating a movable connection of the clamping mechanism to the container via a fixed rotation point or a fixed rotation axis arranged in a jacket area of the container;
the applying including moving the clamping mechanism to a first position of the clamping mechanism on the eye;
manually or automatically aligning the device to an optical axis of the eye;
moving the clamping mechanism of the device to a second position, in which parts of the clamping mechanism are arranged between an eyeball of the eye and a lid of the eye as a result of which the frictionally engaged coupling of the container to the eye is established.

31. The method according to claim 30, further comprising utilizing structure of the clan ping mechanism to block the eyelids from closing.

32. The method according to claim 30, further comprising utilizing structure on the lower edge of the container which has an adhesion effect that minimizes relative movement between the eye and the container.

33. The method according to claim 30, further comprising indenting knobs on an eye contact surface of the container into a conjunctiva of the eye thus minimizing relative movement between the eye in the container.

34. The method according to claim 30, further comprising inserting a contact glass into the container or between the container and the eye or tilling the container with a refractive acting fluid or a refractive acting gel.
